(19) United States
(12) Patent Application Publication
(10) Pub. No.: US 2015/0258047 A1
(43) Pub. Date: Sep. 17, 2015

(54) Therapeutic Compounds

(71) Applicant: AKTHELIA PHARMACEUTICALS, Reyjavik (IS)

(72) Inventors: Rubhana Raqib, Dhaka (BD); Birgitta Agerberth, Stockholm (SE); Gudmundur Hrafn Gudmundsson, Reykjavik (IS); Roger Stromberg, Huddinge (SE)

(21) Appl. No.: 14/723,623

(22) Filed: May 28, 2015

Related U.S. Application Data
(63) Continuation of application No. 14/111,013, filed on Dec. 18, 2013.
(60) Provisional application No. 61/474,100, filed on Apr. 11, 2011.

(51) Int. Cl. A61K 31/192; A61K 31/59

(52) U.S. Cl. CPC A61K 31/192; A61K 31/59

(57) ABSTRACT

The invention relates to compounds which are active as drugs for stimulating the innate antimicrobial peptide system and can be used as antimicrobial drugs. Preferred targets are infections selected from infection of the lung, trachea, urinary tract or kidney, upper GI tract and\or blood. Preferred target pathogens are selected from: Mycobacterium tuberculosis; Pseudomonas bacteria; Haemophilus influenzae; Moraxella catarrhalis. “Preferred” compounds of the invention are. Preferred compounds include 4-phenylbutyric acid or a salt of 4-phenylbutyrate, such as sodium 4-phenylbutyrate, Butyric acid or a salt of butyrate, such as sodium butyrate, or glyceryl tributyrate.

### TECHNICAL FIELD

The invention relates to compounds which are active as drugs for stimulating the innate antimicrobial peptide system and can be used as antimicrobial drugs.

### BACKGROUND ART

Antimicrobial peptides and proteins play an important role in innate host defences and are believed to be particularly important at mucosal surfaces that form the initial barrier between the host and the external environment. Such peptides are found in large quantities in the colonic epithelium. The peptides can be considered as endogenous antibiotics and are widespread in nature as immediate defence effectors. They are mainly stored in vacuoles of granulocytes ready for activation upon stimulation or secreted directly onto mucosal and other surfaces by epithelial cells.

A human antimicrobial peptide has been identified and is referred to as cathelicidin or LL-37, a 37-residue peptide present in neutrophils, epithelial cells and lymphocytes. Both isolated and chemically synthesised LL-37 show antimicrobial activity in vitro.

Certain bacteria have evolved mechanisms to overcome the antimicrobial peptide barrier, such as Shigella bacteria which down-regulate LL-37 expression in the colon epithelium.

WO2009/087474 concerns generally the use of short chain fatty acids (SCFAs) and glycerol esters of SCFAs, and other compounds including vitamin D, for treating, preventing or counteracting microbial infections in animals by stimulating the innate antimicrobial peptide defence system, such as LL-37 in humans. Preferred compounds include phenyl substituted short chain fatty acid derivatives. This publication describes, inter alia, how CAP-18 (the rabbit homologue to LL-37) is induced in the rabbit colonic epithelium following oral administration. The publication further describes the expression of LL-37 in a bronchial epithelial cell line VA10. The publication further describes the cure of rabbits from shigellosis.

WO2008/073174 (GALLO) describes methods and compositions for modulating gene expression and the innate immune response by use of 1,25(OH)₂ vitamin D3 (1,25D3).

That compound is tested alongside non-specific histone deacetylase inhibitors (HDACi) including butyrate or trichostatin A.

US20080038374 (Stahle) describes use of a vitamin D compound, which is able to specifically and directly up-regulate hCAP18, for the manufacturing of a medicament with antimicrobial effect for treatment of conditions deficient in LL-37, such as chronical ulcers, and atopic dermatitis.

Liu et at “Toll-Like Receptor Triggering of a Vitamin D-Mediated Human Antimicrobial Response” 24 Mar. 2006 VOL 311 SCIENCE, pp 1770-1773, describes data which is said to support a link between TLRs and vitamin D-mediated innate immunity and suggest that differences in ability of human populations to produce vitamin D may contribute to susceptibility to microbial infection, such as Mycobacterium tuberculosis.

Hata at al. (2008) “Administration of oral vitamin D induces cathelicidin production in atopic individuals” J ALLERGY GUN IMMUNOL, VOLUME 122, NUMBER 4, described a study in which 14 normal controls and 14 atopic subjects with moderate to severe atopic dermatitis were treated with oral vitamin D3 to see if this could overcome the relative deficiency in induction of cathelicidin in the atopic patients. After supplementation with 4000 IU/d oral vitamin 0 for 21 days, AD lesional skin showed a statistically significant increase in cathelicidin expression.

The synergistic effects of PBA and vitamin D has been demonstrated in vitro in the VA10 cell line in a publication by Steinmann et al (2009) ANTIMICROBIAL AGENTS AND CHEMOTHERAPY (53), 5127-5133.

Martineau at al (Lancet 2011; 377: 242-50) describes a Phase II study of TB patients treated with high dose vitamin D.

Despite the above disclosures, it will be appreciated that the provision of compounds or combinations of compounds for use in enhancing the innate immune response against organisms or diseases not previously identified targeted in this way, or in tissues over and above those previously identified, would provide a contribution to the art.

### DISCLOSURE OF THE INVENTION

As described in the Examples below, the present inventors have demonstrated that compounds described herein can induce LL-37 related peptides systemically, in trachea, lung, kidney and urinary tract. This finding provides for novel therapies not taught or suggested in the prior art.

For example, as described in the Examples below, the present inventors have demonstrated that after oral treatment of Shigella infected rabbits with compounds of the invention, CAP-18 is produced at increased levels in the trachea, and lung relative to untreated, infected animals. Other experiments, including those using IV dosing, herein have demonstrated utility for the compounds in kidney urinary tract, upperGI-tract and blood.

This suggests an increased utility for these compounds in the treatment of infections in these internal organs, over and above the utility previously indicated for infections of the lower GI-tract, by oral dosing, to boost antimicrobial activity (e.g. secretion of LL-37 or defensins, in humans).

US 200210076393 A1 relates to a method for the stimulation of defensin production in eukaryotic cells such as, for example, mammalian cells and various organs, using isoleucine or active isomers or analogs thereof. It further relates to methods for the prevention and treatment of infections and other various disease states and in the stimulation of the immune system in various tissues in which defensins are found. However this publication does not relate to compounds of the type utilised in the present invention.

US20060045912 relates to controlled-release formulations and dosage forms containing 4-phenylbutyric acid sodium salt, or other pharmaceutically acceptable salts, esters or prodrugs, and a controlled release material for use in the treatment of diseases and disorders including neoplastic disorders and neurodegenerative diseases. It refers, inter alia to treatment of kidney cancer and lung cancer. However this publication does not relate to boosting antimicrobial activity in these organs to counter infection therein.

U.S. Pat. No. 5,635,533 relates to compositions and methods of treating anemia, cancer, AIDS, or severe F-chain hemoglobinopathies by administering a therapeutically effective amount of phenylacetate or pharmaceutically acceptable derivatives thereof or derivatives thereof alone or in combination or in conjunction with other therapeutic agents. However this publication does not relate to boosting antimicrobial activity in these organs to counter infection therein.

The microbial targets and diseases targeted by the present invention are preferably as described hereinafter.

The present inventors have shown that Shigella infection causes down-regulation of the antimicrobial peptide CAP-18 in lung and tracheal epithelia. The Shigella associated down-regulation of CAP-18 suggests a functional decline in the innate epithelial barrier of the respiratory system, facilitating invasion by respiratory pathogens. This may partially explain the frequent association of pneumonia with shigellosis.

Thus in one aspect the invention comprises use of the compounds described herein to counteracting bacterial-mediated down-regulation of anti-microbial peptides in the mucosal epithelia of the respiratory tract (e.g. respiratory airways such as the trachea, and the lungs). This may have particular utility in the treatment of secondary respiratory infections that are frequently, and sometimes lethally, associated with dysenteric diarrhoea or the like. Treatment of Acute Respiratory Infections (ARI) forms one aspect of the invention.

The present example demonstrates that treatment with compounds of the invention (for example PBA [phenylbutyric acid] or sodium phenylbutyrate, optionally with vitamin 0) leads to expression of LL-37 in blood macrophages in humans. Furthermore, the same macrophages demonstrate improved efficacy in killing of TB bacteria in vitro.

Thus in one aspect the invention comprises use of the compounds described herein to induce anti-microbial peptides in white blood cells (e.g. macrophages and neutrophils).

This may have utility in the treatment of infections of the blood, for example in immunocompromised patients. Infections may for example be bacterial or viral infections. A particular target identified by the present inventors is the treatment of tuberculosis TB. Thus in one aspect the invention comprises use of the compounds described herein to induce anti-bacterial peptides to inhibit the activity of Mycobacterium tuberculosis bacteria.

U.S. Pat. No. 6,011,000 relates to compositions useful in the treatment and prevention of blood disorders such as anemia, thalassemia and sickle cell disease. Compositions comprise proteins or chemicals that stimulate the specific expression of a globin protein or the proliferation or development of hemoglobin expressing or other myeloid cells. However this publication does not relate to boosting antimicrobial activity in the blood to counter infection therein.

As described in the Examples below, the present inventors have demonstrated the killing of Pseudomonas bacteria by lung epithelial cells in culture after being treated with a compound of the invention.

Thus in one aspect the invention comprises use of the compounds described herein to induce anti-bacterial peptides to inhibit the activity of Pseudomonas bacteria e.g. Pseudomonas aeruginosa. This may have particular utility in the treatment of Pseudomonas infections of the lung.

The inventors have further demonstrated in vitro killing of respiratory pathogens, Haemophilus influenzae and Moraxella catarrhalis by CAP-18 and LL-37.

Thus in one aspect the invention comprises use of the compounds described herein to induce anti-bacterial peptides to inhibit the activity of Haemophilus influenzae and Moraxella catarrhalis.

Another preferred embodiment of the invention is the use of glyceryl tributyrate (TBG) as a therapeutic or prophylactic measure for kidney infections. In another embodiment the invention relates to use of the invention in respect of treatment or prophylaxis of urinary tract infections.

Thus, in particular aspects of the invention, there are provided methods for treatment or prophylaxis of a microbial infection in a patient in need of the same, by administering, preferably orally, to the patient an effective amount of a compound of the invention as described herein. In other embodiments, administration may be intravenous.

Aspects of the invention include a method for treating, preventing or counteracting microbial infections, including bacterial, viral, fungal and parasitic infections (also including infections by bacterial strains resistant to currently used antibiotics), by administering a medicament comprising a secretagogue-effective amount of at least one compound of the invention as defined herein.

In yet a further aspect, the invention provides a pharmaceutical composition for use in the methods described herein e.g. for treating, preventing or counteracting a microbial infection, including the above mentioned types, comprising an active ingredient being at least one compound of the invention, and typically at least one pharmaceutically acceptable excipient.

In yet a further aspect, the invention provides use of compounds of the invention in the preparation of a medicament for use in the methods described herein.

### Compounds of the Invention

Compounds of the invention are those defined by formula I:

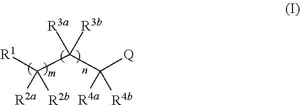

wherein

Q represents —COOH, —COOR⁵, or a pharmaceutically acceptable salt of —COOH;
R¹ represents hydrogen, halide, amino, hydroxyl, carbonyl, a linear or branched substituted or unsubstituted saturated or unsaturated alkyl group with 1 to 10 carbon atoms, or a substituted or unsubstituted aryl group;
R^{2a}, R^{2b}, R^{3a}, R^{3b}, R^{4a} and R^{4b}, if present, each independently represent hydrogen, halide, amino, hydroxyl, carbonyl, a linear or branched substituted or unsubstituted saturated or unsaturated alkyl group with 1 to 10 carbon atoms, or a substituted or unsubstituted aryl group; and/or
R^{3a}, together with an adjacent R^{4a} or R^{2a}, may represent a carbon-carbon π bond; and/or
R^{3b}, together with an adjacent R^{4b} or R^{2b}, may represent a carbon-carbon π bond;
m and n are each independently 0 or 1;
R⁵, if present, represents a linear or branched substituted or unsubstituted saturated or unsaturated alkyl group with 1 to 10 carbon atoms, a substituted or unsubstituted aryl group, a triglyceride moiety —CH₂CH(OC(═O)R⁶)CH₂(OC(═O)R⁷), or a diglyceride moeity —C(═O)OCH₂CH(OC(═O)R⁶)CH₂OH or a salt thereof; and
R⁶ and R⁷, if present, independently represent hydrogen, a linear or branched substituted or unsubstituted saturated or unsaturated alkyl group with 1 to 10 carbon atoms or a substituted or unsubstituted aryl group.

Preferences for these groups are discussed hereinafter.

### Preferred Compounds

“Preferred” compounds of the invention are:
4-phenylbutyric acid or a salt of 4-phenylbutyrate, such as sodium 4-phenylbutyrate (‘PBA’, compound IIa)
Butyric acid or a salt of butyrate, such as sodium butyrate (compound IIb)
2-methyl-3-phenylpropionic acid or a salt of 2-methyl-3-phenylpropionate such as sodium 2-methyl-3-phenylproprionate (IId).
glyceryl tributyrate (‘TBG’, compound IVa)

PBA is a known medicament. For example it has been marketed by Ucyclyd Pharma (Hunt Valley, USA) under the trade name Buphenyl and by Swedish Orphan International (Sweden) as Ammonaps. It has been used to treat urea cycle disorders (Batshaw et al. (2001) J. Pediatr. 138 (1 Suppl): S46-54; discussion S54-5). Scandinavian Formulas, Inc. Sellersville, Pa. supplies sodium phenylbutyrate worldwide for clinical trials. Sodium phenylbutyrate is also under investigation for the treatment of some sickle-cell disorders (Blood Products Plasma Expanders and Haemostatics) and for use as a potential differentiation-inducing agent in malignant glioma and acute myeloid leukaemia. It has also been investigated in respect of cystic fibrosis pathology due to its capacity to traffic DeltaF508-cystic fibrosis transmembrane conductance regulator (CFTR) to the cell membrane and restore CFTR chloride function at the plasma membrane of CF lung cells in vitro and in vivo (Roque et al. J Pharmacol Exp Ther. 2008 September; 326(3):949-56. Epub 2008 Jun. 23). It is believed in the literature that phenylbutyrate is a prodrug which is metabolized in the body by beta-oxidation to phenylacetate.

“More preferred” compounds of the invention are:
4-phenylbutyric acid or a salt of 4-phenylbutyrate, such as sodium 4-phenylbutyrate (‘PBA’, compound Ha)
2-methyl-3-phenylpropionic acid or a salt of 2-methyl-3-phenylpropionate such as sodium 2-methyl-3-phenylproprionate (IId).
glyceryl tributyrate (‘TBG’, compound IVa)

“Most preferred” compounds of the invention are:
4-phenylbutyric acid or a salt of 4-phenylbutyrate, such as sodium 4-phenylbutyrate (‘PBA’, compound IIa)

Thus any aspect or embodiment of the invention is preferably performed using these more or most preferred compounds.

### Treatment

The term “treatment,” as used herein in the context of treating a disorder, pertains generally to treatment and therapy, whether of a human or an animal, in which some desired therapeutic effect is achieved, for example, the inhibition of the progress of the disorder, and includes a reduction in the rate of progress, a halt in the rate of progress, alleviation of symptoms of the disorder, amelioration of the disorder, and cure of the disorder.

The term “patient” should thus be interpreted to include animals, and the methods and compositions of the present invention will be understood to have utility in veterinary and animal husbandry applications for companion animals, farm animals, and ranch animals. These applications include but are not limited to treating, preventing or counteracting microbial diseases and conditions in dogs, cats, cows, horses, deer and poultry including hen, turkey ducks, geese; as well as in household pets such as birds and rodents. For large animals, a suitable dose can be larger than the above mentioned amounts.

Treatment as a prophylactic measure (i.e., prophylaxis) is also included. For example, use with patients who have not yet developed the disorder, but who are at risk of developing the disorder, is encompassed by the term “treatment.”

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

### Combination Therapies

The term “treatment” includes combination treatments and therapies, in which two or more treatments or therapies are combined, for example, sequentially or simultaneously. For example, the compounds described herein may in any aspect and embodiment also be used in combination therapies, e.g. in conjunction with other agents (an example is PBA and Vitamin D, or polyamines (such as spermidine, spermine, putrescine; see WO2009/087474).

The agents (i.e. the compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g. 1, 2, 3, 4 or more hours apart, or even longer periods apart where required), the precise dosage regimen being commensurate with the properties of the therapeutic agent(s) as described herein, including their synergistic effect.

The agents (i.e. the compound described here, plus one or more other agents) may be formulated together in a single dosage form, or alternatively, the individual agents may be formulated separately and presented together in the form of a kit, optionally with instructions for their use.

A preferred combination is PBA and Vitamin D, for example in treating TB in patients, for example who may be HIV positive. In support of this a Phase II study is underway in respect of the use of this combination as an adjunct to classical (or conventional) therapy in the treatment of TB, for example using conventional antibiotics, for example to reduce treatment times.

Another preferred combination is PBA and Vitamin D, for example for the treatment of Pseudomonas lung infection.

As noted above, in certain aspects, it may be preferred to use the compounds described herein in conjunction with a known antibiotic, as follows:

(1) acute administration to the patient of an antibiotic for preferably 1, or 2, days with or without a compound of formula (I); followed by,
(2) administration to the patient of an effective amount of a compound of formula (I) for a further 2, 3, 4, 5 or more days.

Such a regime may have benefits in minimising the development of antibiotic resistance in the pathogen to be targeted.

### Dosages

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

The compounds of the present invention exhibit an antimicrobial effect by stimulating the innate antimicrobial peptide defense system.

Thus an effective amount in the present context would be one which is sufficient to demonstrate antimicrobial activity in vivo e.g. by stimulating (e.g. de-repressing or inhibiting down-regulation of) synthesis of the cathelicidin LL-37 or other naturally occurring antibiotic peptide or protein e.g. a defensin. Stimulation may be towards, equal to, or above basal levels (i.e. normal levels in the absence of the infection).

By the term “antimicrobial activity” as used herein, is meant the ability to inhibit the growth of or actually kill a population of microbes which can be bacteria, viruses, protozoa or fungal microbes. Thus “antimicrobial activity” should be construed to mean both microbistatic as well as microbicidal activities. Antimicrobial activity should also be construed to include a compound which is capable of inhibiting infections, i.e. disease-causing capacity of microbes. Generally the use of the present invention will be such as to lead to secretion of the relevant peptide onto an epithelial surface.

In the present invention the compounds are administered orally. As described in the Examples below, the present inventors have demonstrated that compounds described herein can induce therapeutically relevant concentrations of antibacterial peptides in diverse tissues following oral administration of even relatively low dosages. This finding therefore opens the possibility of treating infections not previously envisaged as being treatable in this way.

It will be apparent that the invention envisages use of the compounds described herein at dosages which would not achieve a “minimum inhibitory concentration” (MIC) required for a direct inhibitory effect on the targeted pathogen.

For example, as explained below, LL-37 is expressed in human blood cells when PBA is administered as 500 mg tablets twice daily in combination with Vitamin D (information on low dose).

Preferred dosages and dosage forms are described in more detail below.

A preferred daily dosage of PBA may be

between 500 mg and 2000 mg
more preferably 750 to 1500 mg
more preferably 750 to 1250 mg
more preferably about 900 to 1100
more preferably about 1000 mg/day, optionally with vitamin D3.

In each case dosages can be split into 1, 2, 3, or 4 doses per day. For example 2 or 3×250 mg/day, 2×500 mg/day or 2×1000 mg/day

A preferred daily dosage of TBG may be between 1000 mg and 4000 mg; between 2000 mg and 4000 mg; more preferably 3000 to 4000 mg; more preferably about 3000 or 3500 or 4000 mg, optionally with vitamin D3.

In each case dosages can be split into 1, 2, 3, or 4 doses per day. For example 2 or 3×500 mg/day, 2×1000 mg/day and so on.

A preferred daily dose for IV administration, based on the effective intravenous administration in rabbits described below, is between 200 and 700 mg of sodium butyrate; between 300 and 550 mg of sodium butyrate; more preferably 400 to 500 mg; more preferably about 450 mg of sodium butyrate.

A preferred daily dose for IV administration, is: between 500 and 950 or 1000 mg of sodium phenylbutyrate; between 600 and 850 mg of sodium phenylbutyrate; more preferably 650 to 800 mg; more preferably about 750 mg of sodium phenylbutyrate.

Corresponding preferred weight\molar amounts for other compounds of the invention can be calculated by those skilled in the art based on the disclosure herein.

In each case intravenous dosages can be split into 1, 2, 3, or 4 doses per day. Dosing twice daily may be preferred.

Dosages for Vitamin D may be of the order of 1000-10 000 IU daily.

### Dosage Forms

The compound of the invention is preferably administered in an oral dosage form such as, but not limited to, a tablet, a capsule, a solution, a suspension, a powder, a paste, an elixir, and a syrup.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Other administration forms are also useful, these include but not are limited to topical administration forms, which are in particular useful against infections of the skin, these include for example creams, oils, lotions, and ointments. Yet further dosage forms include dosage forms for delivery to the respiratory system including the lungs, such as aerosols and nasal spray devices or by rectal anema (as done in patients with shigellosis). However, as described herein, the present inventors have demonstrated a systemic effect (as evidenced in kidney, trachea and lung) following oral administration. Thus direct delivery to targeted internal organs is not necessary. Intravenous administration showed a similar effect to the oral NaB dose in inducing CAP-18 in colon, rectum and lung confirming the systemic effect.

### Functional Foods

It will also be appreciated, in particular when it is desired to administer a large amount of active compound, such as, in the range of 1-25 g that the compounds of the invention can be (isolated and then) formulated and comprised in functional food or feed products. Such functional food products include but are not limited to fermented food products including fermented bean products, e.g. soy bean products such as tempeh, products from fermented oat, germinated barley, and similar products. Such products, generally produced by microbial fermentation which breaks down betaglucans, will have a natural content of short chain fatty acids that can boost the effect of the compounds of the present invention. The form of functional food product in accordance with the invention can be any form suitable for the chosen food type, including crackers, pastry, spread or paste, a purée, a jelly, a yoghurt, a drink concentrate, or any other suitable food product in which the selected active compound(s) can be readily formulated in.

### Preferences of Q

In Formula I described herein before Q may be —COOH, a pharmaceutically acceptable salt of —COOH or —COOR⁵.

In certain preferred embodiments, Q represents a pharmaceutically acceptable salt of —COOH. Pharmaceutically acceptable salts of carboxylic acids are known in the art.

Preferably Q represents a pharmaceutically acceptable metal ion salt of —COOH. Preferably, the pharmaceutically acceptable metal ion is Na⁺ or K⁺.

Particularly preferred compounds where Q is a salt of —COOH are sodium 4-phenylbutyrate (IIa), sodium butyrate (IIb), sodium 2,2-dimethylbutyrate (IIc) and sodium 2-methyl-3-phenylproprionate (IId).

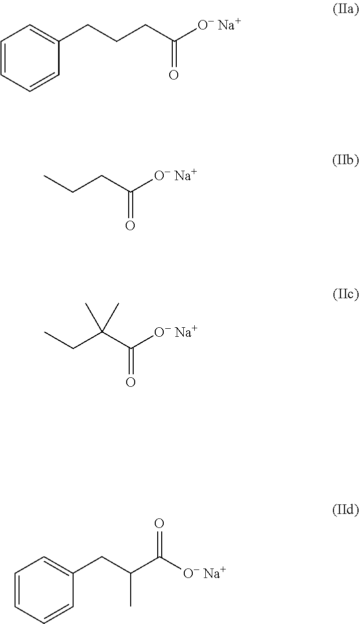

In other preferred embodiments of the present invention, Q represents —COOR⁵.

R⁵ may be a linear or branched substituted or unsubstituted saturated or unsaturated alkyl group with 1 to 10 carbon atoms, a substituted or unsubstituted aryl group, a triglyceride moiety —CH₂CH(OC(═O)R⁶)CH₂(OC(═O)R⁷), or a diglyceride moiety —C(═O)OCH₂CH(OC(═O)R⁶)CH₂OH or a salt thereof.

When R⁵ is an alkyl group, the alkyl group preferably has 1 to 5 carbon atoms. More preferably, the alkyl group is selected from methyl and ethyl.

Preferably, R⁵ is an alkyl group with 1 to 10 carbon atoms substituted with an aryl group. More preferably, R⁵ is a methyl group substituted with a phenyl group, in other words, R⁵ forms a benzyl group.

When R⁶ is an aryl group, the aryl group is preferably unsubstituted or substituted phenyl.

Preferably R⁵ forms a triglyceride moeity —CH₂CH(OC(═O)R⁶)CH₂(OC(═O)R⁷) or a diglyceride moiety —C(═O)OCH₂CH(OC(═O)R⁶)CH₂OH or salt thereof.

If R⁵ forms a triglyceride moeity, the compounds of the invention are of the following general formula (IIIa):

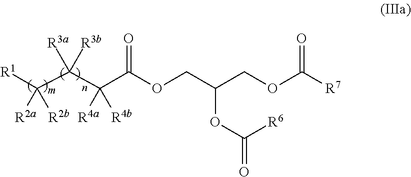

If R⁵ forms a diglyceride moeity, the compounds of the invention are of the following general formula (IIIb):

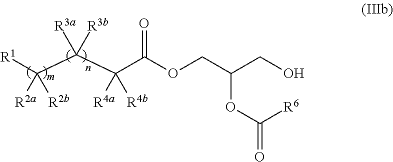

or salt thereof,

R⁶ and R⁷ may be independently hydrogen, a linear or branched substituted or unsubstituted saturated or unsaturated alkyl group with 1 to 10 carbon atoms or a substituted or unsubstituted aryl group.

Preferably R⁶ and R⁷ are independently selected from H and an alkyl group with 1 to 5 carbon atoms.

Embodiments of particular interest include glyceryl tributyrate (IVa) and glyceryl tripropionate (IVb):

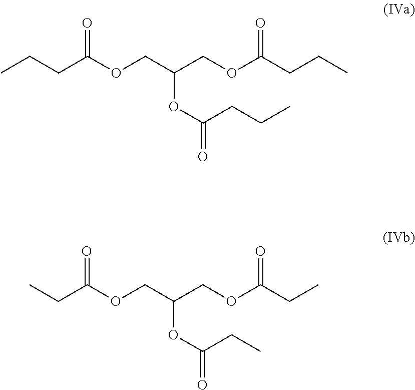

Other preferred embodiments include glyceryl tributyrate wherein one or more of the butyrate acyl chains are substituted with phenyl, e.g. 1-butanoyloxy-3-(4′-phenylbutanoyloxyl)propan-2-yl butanoate, 1,3-(4′,4″-diphenyl)-di(butanoyloxy)propan-2-yl butanoate, and 1,3-di(butanoyloxy)propan-2-yl-4-phenylbutanoate.

### Preferences of R¹

In some preferred embodiments, R¹ is selected from Hand a substituted or unsubstituted saturated or unsaturated alkyl group with 1 to 10 carbon atoms.

When R¹ is an alkyl group, the alkyl group preferably has 1 to 5 carbon atoms. More preferably, the alkyl group is selected from methyl and ethyl.

Preferably, R¹ is an alkyl group with 1 to 10 carbon atoms substituted with an aryl group. More preferably, R¹ is a methyl group substituted with a phenyl group, in other words, R¹ forms a benzyl group.

In other preferred embodiments, R¹ is aryl, preferably phenyl or substituted phenyl.

In particularly preferred embodiments R¹ is an optionally substituted aryl group, such as phenyl and Q is a salt of —COOH. According to these embodiments, the compounds may be represented by general formula (V):

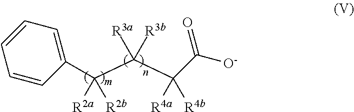

Preferred butyric acid derivatives (butyrates) are therefore of general formula (Va):

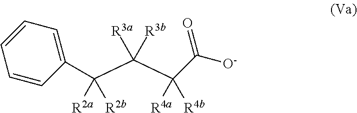

preferred proprionic acid derivates (proprionates) are of general formula (Vb):

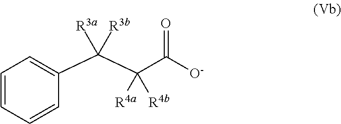

And preferred acetic acid derivatives (acetates) are of general formula Vc:

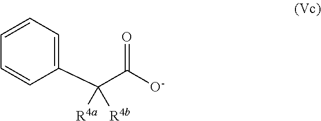

### Preferences of Chain Length (I.e. of m and n)

m and n may each be 0. The resulting compounds have a chain length between Q and R¹ of 1 and may be described as acetic acid or acetate derivatives of general formula (VIa):

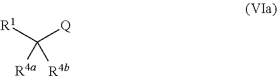

Alternatively, m is 0 and n is 1. The resulting compounds have a chain length between Q and R¹ of 2 and may be described as proprionic acid or proprionate derivatives of general formula (VIb):

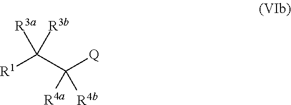

Proprionic acid or proprionate derivatives are also formed when m is 1 and n is 0.

Preferably m and n are each 1. The resulting compounds have a chain length between Q and R¹ of 3 and may be described as butyric acid or butyrate derivatives of general formula (VIc):

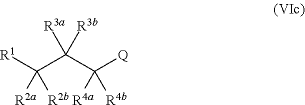

When m is 0, R^{2a} and R^{2b} are not present, and when n is 0, R^{3a} and R^{3b} are not present.

Substituents α to the Carboxylic Acid or Carboxylate, Q (R^{4a} and/or R^{4b})

R^{4a} and R^{4b} are preferably each independently selected from hydrogen, a linear or branched substituted or unsubstituted saturated or unsaturated alkyl group with 1 to 10 carbon atoms, or a substituted or unsubstituted aryl group.

At least one of R^{4a} and R^{4b} may be selected from hydrogen and an alkyl group having from 1 to 10 carbon atoms, the alkyl group being preferably methyl or ethyl. In some embodiments, R^{4a} and R^{4b} may both be alkyl, but it is preferred that at least one of R^{4a} and R^{4b} is hydrogen.

In particular, the following compounds are useful in accordance with the invention: 4-phenylbutyric acid, 3-phenylbutyric acid, 2-phenylbutyric acid, 3-phenylpropionic acid, 2-phenylpropionic acid, 2-methyl-3-phenylpropionic acid, 2-methyl-4-phenylbutyric acid, or a pharmaceutically acceptable salt of any of said compounds, methyl 4-phenylbutyrate, ethyl 4-phenylbutyrate, methyl 3-phenylbutyrate, ethyl 3-phenylbutyrate, methyl 2-phenylbutyrate, ethyl 2-phenylbutyrate, methyl 3-phenylpropionate, ethyl 3-phenylpropionate, methyl 2-phenylpropionate, ethyl 2-phenylpropionate, methyl 2-methyl-3-phenylpropionate, ethyl 2-methyl-3-phenylpropionate, methyl 2-methyl-4-phenylbutyrate, and ethyl 2-methyl-4-phenylbutyrate.

Metabolites of these compounds may also be useful in the invention, in particular phenyl acetate and 4-phenyl butyrate.

### Substituents β to the Carboxylic Acid or Carboxylate, Q (Where Present)

In some embodiments, one or both of R³ and R^{3b} (or R^{2a} and R^{2b} if n is 0 and m is 1) may optionally be hydroxyl. This may be preferred where it is desired that the compound of the invention have increased resistance to metabolism such as beta oxidation, and hence in principle a longer half-life.

In another aspect of the present invention provides a composition of a compound as defined by formula I and a Vitamin D compound or salt thereof for use in combination in a method of treating, preventing or counteracting microbial infections in humans and animals by stimulating the innate antimicrobial peptide defense system,

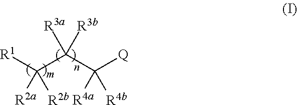

wherein
Q represents —COOH, —COOR⁵, or a pharmaceutically acceptable salt of —COOH;
R¹ represents hydrogen, halide, amino, hydroxyl, carbonyl, a linear or branched substituted or unsubstituted saturated or unsaturated alkyl group with 1 to 10 carbon atoms, or a substituted or unsubstituted aryl group;
R^{2a}, R^{2b}, R^{3a}, R^{3b}, R^{4a} and R^{4b}, if present, each independently represent hydrogen, halide, amino, hydroxyl, carbonyl, a linear or branched substituted or unsubstituted saturated or unsaturated alkyl group with 1 to 10 carbon atoms, or a substituted or unsubstituted aryl group; and/or
R^{3a}, together with an adjacent R^{4a} or R^{2a}, may represent a carbon-carbon π bond; and/or
R^{3b}, together with an adjacent R^{3b} or R^{2b}, may represent a carbon-carbon π bond;
m and n are each independently 0 or 1;
R⁵, if present, represents a linear or branched substituted or unsubstituted saturated or unsaturated alkyl group with 1 to 10 carbon atoms, a substituted or unsubstituted aryl group, a triglyceride moiety —CH₂CH(OC(═O)R⁶)CH₂(OC(═O)R⁷), or a diglyceride moeity —C(═O)OCH₂CH(OC(═O)R⁶)CH₂OH or a salt thereof;
R⁶ and R⁷, if present, independently represent hydrogen, a linear or branched substituted or unsubstituted saturated or unsaturated alkyl group with 1 to 10 carbon atoms or a substituted or unsubstituted aryl group

Vitamin D compounds are a group of fat-soluble secosteroids, and the group includes Vitamin D₁, Vitamin D₂, Vitamin D₃, Vitamin D₄ and Vitamin D₅.

Preferably the composition includes a Vitamin D compound selected from one of Vitamin D₂ and Vitamin D₃.

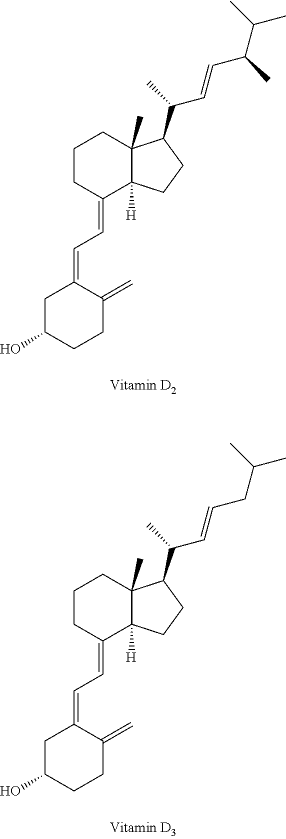 [SMILES: C=C1CC[C@H](O)C/C1=C/C=C1\CCC[C@]2(C)[C@@H]([C@H](C)/C=C/[C@H](C)C(C)C)CC[C@@H]12 ; C=C1CC[C@H](O)C/C1=C/C=C1\CCC[C@]2(C)[C@@H]([C@H](C)/C=C/CC(C)C)CC[C@@H]12]

The optional and preferred features of the first aspect of the invention apply equally to further aspects. In particular, the preferred compounds of formula (I) in the first aspect of the invention are preferred compounds of the second aspect of the invention relating to the composition of a compound of formula (I) and a Vitamin D compound.

### DEFINITIONS AND FURTHER PREFERENCES

### Alkyl:

As used herein the term “alkyl”, unless otherwise specified, refers to a C₁₋₁₀ alkyl group, that is to say a monovalent moiety obtained by removing a hydrogen atom from a hydrocarbon compound having from 1 to 10 carbon atoms, which may be aliphatic or alicyclic, or a combination thereof, which may be linear or branched, and which may be saturated, partially unsaturated, or fully unsaturated. In certain instances C₁₋₄, C₁₋₅, C₁₋₆ or C₁₋₇ alkyl groups may be preferred.

Examples of saturated linear C₁₋₁₀ alkyl groups include, but are not limited to, methyl, ethyl, n-propyl, n-butyl, n-pentyl (amyl) and n-hexyl.

Examples of saturated branched C₁₋₁₀ alkyl groups include, but are not limited to, iso-propyl, iso-butyl, sec-butyl, tert-butyl, and neo-pentyl.

Examples of saturated alicyclic C₁₋₁₀ alkyl groups (which may also be referred to as “C₃₋₁₀ cycloalkyl” groups) include, but are not limited to, groups such as cyclopropyl, cyclobutyl, cyciopentyl, and cyclohexyl, as well as substituted groups (e.g., groups which comprise such groups), such as methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

Unsaturated alkyl groups contain one or more double or triple bonds i.e. one or more carbon-carbon π bonds. Examples of unsaturated C₁₋₁₀ alkyl groups which have one or more carbon-carbon double bonds (also referred to as “C₂₋₁₀alkenyl” groups) include, but are not limited to, ethenyl (vinyl, —CH═CH₂), 2-propenyl (ally, —CH—CH═CH₂), isopropenyl (—C(CH₃)═CH₂), butenyl, pentenyl, and hexenyl.

Examples of unsaturated C₁₋₁₀ alkyl groups which have one or more carbon-carbon triple bonds (also referred to as “C₂₋₁₀ alkynyl” groups) include, but are not limited to, ethynyl (ethinyl) and 2-propynyl (propargyl).

Examples of unsaturated alicyclic (carbocyclic) C₁₋₁₀ alkyl groups which have one or more carbon-carbon double bonds (also referred to as “C₃₋₁₀cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl and cyclohexenylmethyl.

### Aryl:

As used herein the term “aryl”, unless otherwise specified, refers to a C₅₋₂₀ aryl group, that is to say a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups”, in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), naphthacene (C₁₈), and pyrene (C₁₆).

Examples of aryl groups which comprise fused rings, one of which is not an aromatic ring, include, but are not limited to, groups derived from indene and fluorene.

Alternatively, the ring atoms may include one or more heteroatoms, including but not limited to oxygen, nitrogen, and sulphur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine), triazine, tetrazole, and oxadiazole (furazan).

Examples of C₅₋₂₀ heteroaryl groups which comprise fused rings, include, but are not limited to, C₉ heterocyclic groups derived from benzofuran, isobenzofuran, indole, isoindole, purine (e.g., adenine, guanine), benzothiophene, benzimidazole; C₁₀ heterocyclic groups derived from quinoline, isoquinoline, benzodiazine, pyridopyridine, quinoxaline; C₁₃ heterocyclic groups derived from carbazole, dibenzothiophene, dibenzofuran; C₁₄ heterocyclic groups derived from acridine, xanthene, phenoxathiin, phenazine, phenoxazine, phenothiazine.

### Optional Substitution:

The above alkyl and aryl groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

C₁₋₇ alkoxy: —OR, wherein R is a C₁₋₇ alkyl group. Examples of C₁₋₇ alkoxy groups include, but are not limited to, —OCH₃ (methoxy), —OCH₂CH₃ (ethoxy) and —OC(CH₃)₃ (tert-butoxy).

Oxo (keto, -one): ═O; carbonyl (>C═O). Examples of cyclic compounds and/or groups having, as a substituent, an oxo group (═O) include, but are not limited to, carbocyclics such as cyclopentanone and cyclohexanone; heterocyclics, such as pyrone, pyrrolidone, pyrazolone, pyrazolinone, piperidone, piperidinedione, piperazinedione, and imidazolidone; cyclic anhydrides, including but not limited to maleic anhydride and succinic anhydride; cyclic carbonates, such as propylene carbonate; imides, including but not limited to, succinimide and maleimide; lactones (cyclic esters, —O—C(═O)— in a ring), including, but not limited to, β-propiolactone, γ-butyrolactone, δ-valerolactone, and ε-caprolactone; and lactams (cyclic amides, —NH—C(═O)— in a ring), including, but not limited to, β-propiolactam, γ-butyrolactam (2-pyrrolidone), δ-valerolactam, and ε-caprolactam.

Imino (imine): ═NR, wherein R is an imino substituent, for example, hydrogen, C₁₋₇ alkyl group, a C₃₋₂₀heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Amido (carbamoyl, carbamyl, aminocarbonyl, carboxamide): —C(═O)NR^{N1}R^{N2}, wherein R^{N1} and R^{N2} are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R^{N1} and R^{N2}, together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR^{A1}C(═O)R^{A2}, wherein R^{A1} is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R^{A2} is an acyl substituent, for example, a C₁₋₇ alkyl group, a C₃₋₂₀ heterocyclyl group, or a C₅₋₂₀ aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R^{A1} and R^{A2} may together form a cyclic structure, as in, for example, succinimidyl, maleimidyl and phthalimidyl:

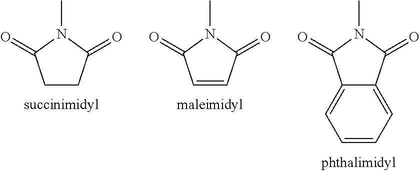

Acylureido: —N(R^{U1})C(O)NR^{U2}C(O)R^{A3} wherein R^{U1} and R^{U2} are independently ureido substituents, for example, hydrogen, a C₁₋₇ alkyl group, a C₃₋₂₀ heterocyclyl group, or a C₅₋₂₀ aryl group, preferably hydrogen or a C₁₋₇ alkyl group. R^{A3} is an acyl group as defined for acyl groups. Examples of acylureido groups include, but are not limited to, —NHCONHC(O)H, —NHCONMeC(O)H, —NHCONEtC(O)H, —NHCONMeC(O)Me, —NHCONEtC(O)Et, —NMeCONHC(O)Et, —NMeCONHC(O)Me, —NMeCONHC(O)Et, —NMeCONMeC(O)Me, —NMeCONEtC(O)Et, and —NMeCONHC(O)Ph.

Carbamate: —NR^{N1}—C(O)—OR^{O2} wherein R^{N1} is an amino substituent as defined for amino groups and R^{O2} is an ester group as defined for ester groups. Examples of carbamate groups include, but are not limited to, —NH—C(O)—O-Me, —NMe-C(O)—O-Me, —NH—C(O)—O-Et, —NMe-C(O)—O-t-butyl, and —NH—C(O)—O—Ph.

Thioamido (thiocarbamyl): —C(═S)NR^{N1}R^{N2}, wherein R^{N1} and R^{N2} are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Tetrazolyl: a five membered aromatic ring having four nitrogen atoms and one carbon atom,

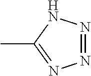

Amino: —NR^{N1}R^{N2}, wherein R^{N1} and R^{N2} are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇alkyl group, or, in the case of a “cyclic” amino group, R^{N1} and R^{N2}, taken together with the nitrogen atom to which they are attached, form a heterocyclic ring having from 4 to 8 ring atoms. Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇ alkyl group.

Amidine: —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. An example of an amidine group is —C(═NH)NH₂.

Carbazoyl (hydrazinocarbonyl): —C(O)—NN—R^{N1} wherein R^{N1} is an amino substituent as defined for amino groups. Examples of azino groups include, but are not limited to, —C(O)—NN—H, —C(O)—NN-Me, —C(O)—NN-Et, —C(O)—NN—Ph, and —C(O)—NN—CH₂—Ph.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃, —S(═O)₂C₄F₉ (nonaflyl), —S(═O)₂CH₂CF₃ (tresyl), —S(═O)₂Ph (phenylsulfonyl), 4-methylphenylsulfonyl (tosyl), 4-bromophenylsulfonyl (brosyl), and 4-nitrophenyl (nosyl).

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ and —OS(═O)₂CH₂CH₃.

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfamino: —NR^{N1}S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfinamino: —NR^{N}'S(═O)R, wherein R^{N1} is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Sulfamyl: —S(═O)NR^{N1}R^{N2}, wherein R^{N1} and R^{N2} are independently amino substituents, as defined for amino groups. Examples of sulfamyl groups include, but are not limited to, —S(═O)NH₂, —S(═O)NH(CHₐ), —S(═O)N(CH₃)₂, —S(═O)NH(CH₂CH₃), —S(═O)N(CH₂CH₃)₂, and —S(═O)NHPh.

Sulfonamino: —NR^{N1}S(═O)₂R, wherein R^{N1} is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅. A special class of sulfonamino groups are those derived from sultams—in these groups one of R¹ and R is a C₅₋₂₀ aryl group, preferably phenyl, whilst the other of R¹ and R is a bidentate group which links to the C₅₋₂₀ aryl group, such as a bidentate group derived from a C₁₋₇ alkyl group. Examples of such groups include, but are not limited to:

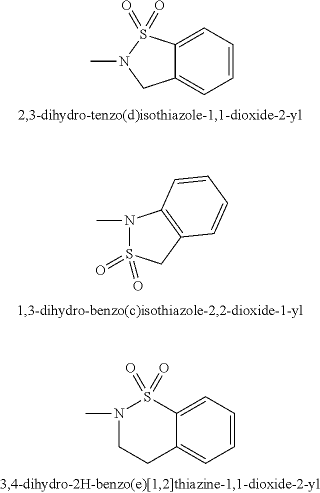

Phosphoramidite: —OP(OR^{P1})—NR^{P2}₂, where R^{P1} and R^{P2} are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR^{P1})—NR^{P2}₂, where R^{P1} and R^{P2} are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

In many cases, substituents may themselves be substituted. For example, a C₁₋₇ alkoxy group may be substituted with, for example, a C₁₋₇ alkyl (also referred to as a C₁₋₇ alkyl-C₁₋₇alkoxy group), for example, cyclohexylmethoxy, a C₃₋₂₀ heterocyclyl group (also referred to as a C₅₋₂₀aryl-C₁₋₇ alkoxy group), for example phthalimidoethoxy, or a C₅₋₂₀ aryl group (also referred to as a C₅₋₂₀aryl-C₁₋₇alkoxy group), for example, benzyloxy.

Preferred substituents for an aryl or alkyl group may include C₁₋₁₀ alkyl groups, C₅₋₂₀ aryl groups, hydroxyl, C₁₋₇alkoxy groups, nitro, amino, substituted amino (—NR^{N1}R^{N2} as defined above) and halides.

### Isomers, Salts, Solvates, and Protected Forms

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

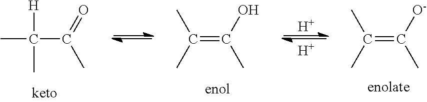

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₂); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO.).

For example, a carboxylic acid group may be protected as an ester for example, as: an C₁₋₇ alkyl ester (e.g. a methyl ester; a t-butyl ester); a C₁₋₇haloalkyl ester (e.g., a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Prodrugs

It is contemplated that some of the active compounds of the invention act in the form of prodrugs. In other words, the compounds are metabolised in the body to the active form. Among these compounds are esters such as glyceryl tributyrate, glyceryl tripropionate, glyceryl tri(4-phenylbutyrate) and methyl 4-phenylbutyrate.

### Vitamin D

Where the term “Vitamin D” is used herein, it is used in a broad sense to encompass Vitamin D3 (or “1,25 D3”) and its hormonally active forms, to include compounds which are structurally similar to vitamin D3. Many of these compounds are recognized and comprise a large number of natural precursors, metabolites, as well as synthetic analogs of the hormonally active 1,25-dihydroxyvitamin D3 (1α25 (OH)₂D3). This language is intended to include vitamin D3, or an analog thereof, at any stage of its metabolism, as well as mixtures of different metabolic forms of vitamin D3 or analogs thereof.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. CAP-18 immunoreactivity in the epithelial lining of different organs in (A) Healthy rabbits, Shigella-infected rabbits and infected rabbits treated with NaB, TBG or PBA (B) healthy rabbits treated with NaB, TBG or PBA. Quantification of immunoreactive area relative to the total cell area of the tissue section was done by a computerized image analysis technique, and the results are expressed as ACIA score, i.e., the total positively stained area×total mean intensity (1-256 levels/per pixel) of the positive area divided by total cell area. One-way ANOVA was used in comparing data between healthy and infected/infected treated rabbits, between infected and infected treated rabbits, and between healthy and healthy-treated rabbits. The differences were significant when P<0.05; ‡ significant when comparing with healthy, * significant when comparing with infected.

FIG. 2. Butyrate concentration in healthy rabbit serum after oral treatment with NaB. Serum was collected from healthy rabbits at different time points after oral treatment with a single 0.14 mmol dose of NaB (15.6 mg). Four analyses (duplicate analyses of two samples) of serum from a NaB treated rabbit are shown, where the concentrations are calculated from two separate standard curves (one for each duplicate). NaB: Sodium butyrate.

FIG. 3. Expression of LL-37 peptide or pro-peptide measured by ELISA in macrophages from blood of healthy volunteers after administration 5000 IU vitamin D and PBA, 500 mg twice or 1000 mg once daily.

FIG. 4. Expression of LL-37 mRNA measured by rtPCR in blood macrophages from healthy volunteers after administration 5000 IU vitamin D and PBA, 500 mg twice or 1000 mg once daily.

FIG. 5. The number of CFU (colony forming units) harvested from co-cultures of blood macrophages collected at day 1, 4 or 8 of the dosing regimen and M. tuberculosis bacteria. n=3 in each group. The order of the bars (left to right) follows the key (top to bottom).

FIG. 6. M. tuberculosis CFUs expressed as average percentages of the CFUs found for each individual at the sampling times indicated in the regimen (at day 1, 4 and 8). The CFUs at day 1 are set as 100%. The bacteria were harvested from co-cultures of blood macrophages collected at day 1, 4 or 8 of the dosing regimen and the number of CFUs was determined. n=3 in each group. The order of the bars (left to right) follows the key (top to bottom).

FIG. 7. Visual assessment of survival of VA10 cells in the presence of low infectivity Pseudomonas aeruginosa 0-1 bacteria (PAO1). After cultivating for 6 days the cells were stimulated for 48 h with 4-PBA, VitD, and 4-PBA+VitD. Subsequently the cells were stimulated for 24 h with 10 MOI PAO1 and inspected under the microscope.

FIG. 8. CAMP expression in VA10 cells after 48 h stimulation with 4 mM 4-PBA, 20 nM VitD, and 4 mM 4-PBA+20 nM VitD and subsequent 24 h stimulation with low infectivity freeze-thawed PAO1 suspension (PAO1 has been frozen in DMEM at −25° C. for a week. The majority of bacteria have been inactivated.)

FIG. 9. DEFB-1 expression in VA10 cells after 48 h stimulation with 4 mM 4-PBA, 20 nM VitD, and 4 nM 4-PBA+20 nM VitD and subsequent 24 h stimulation with low infectivity freeze-thawed PAO1 suspension (PAO1 has been frozen in DMEM at −25° C. for a week. The majority of bacteria have been inactivated.)

### EXAMPLES

### Example 1

### Systemic Induction of LL-37 (Trachea, Lung, Kidney) in the Rabbit after Oral or Intravenous Administration of PBA

### Systemic Effects of Butyrate, Phenylbutyrate and Tributyrate Glyceryl Ester in Shigella-Infected Rabbits

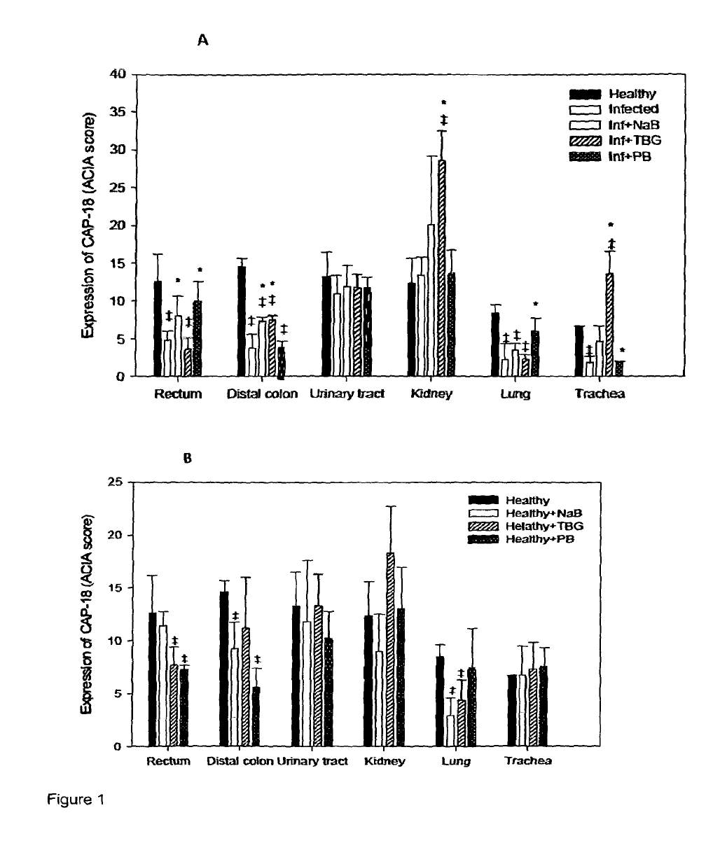

After oral administration of NaB (sodium butyrate), TBG (tributyryl glycerol) and PBA (Phenylbutyric acid/Sodium phenylbutyrate) to rabbits a systemic stimulation of CAP-18 (protein) expression in the shigellosis infected rabbit was observed as evidenced by quantitative assessment of immunostaining in tissue sections from the rectum, distal colon, kidney, lung and trachea relative to samples from untreated, infected rabbits (FIG. 1A). While butyrate and PBA do not cause increase in CAP-18 in the organs of healthy rabbits, TBG causes increased levels of the peptide in the kidney of non-infected rabbits (FIG. 1B), suggesting a preventive treatment for kidney infections.

It has also been demonstrated that significant down-regulation of CAP-18 mRNA in the epithelia of lung and trachea in addition to rectum and colon was detected in Shigella infected rabbits compared to healthy rabbits (Table 1). NaB, TBG or PBA resulted in reduced clinical illness and in up-regulation of CAP-18 peptide in the mucosal epithelia of these organs. Anti-Shigella activity in stool extracts from PBA, TBG and NaB-treated rabbits was higher compared to infected or healthy rabbits. This activity was partially blocked by specific antiserum against CAP-18, demonstrating a contribution of CAP-18 to this anti-Shigella activity. The results suggest a novel mechanism to restore mucosal immunity by counteracting bacteria mediated downregulation of CAP-18 in the mucosal epithelia of the intestinal and the respiratory tracts by butyrate derivatives. Interestingly, due to their systemic effects this treatment could mediate protection from secondary respiratory infections that frequently is the lethal cause in dysenteric diarrhoea.

TABLE 1
CAP-18 gene expression in the tissue specimens from various organs of Shigella-infected
rabbits treated with or without different substances and in healthy control rabbits.

| | Healthy rabbits | Infected rabbits | | Infected rabbits treated with | | | | | |
|---|---|---|---|---|---|---|---|---|---|
| Tissues | (n = 5) | (n = 5) | | NaB (n = 5) | | TBG (n = 5) | | PB (n = 5) | |
| SE | | | | | | | | | |
| Rectum | 0.77 (0.10-1.44) | 0.93 | (0.10-1.76) | 0.3 | (0.06-0.54) | 0.4 | (0.11-0.68) | 0.18 | (0.16-0.20) |
| Distal Colon | 9.3 (4.7-16.1) | 22.2 | (14.3-31.3) | 5.0 | (2.29-6.94)^{b} | 2.8 | (0.72-18.5) | 2.4 | (0.78-29.0) |
| LP | | | | | | | | | |
| Rectum | 0.18 (0.12-0.52) | 0.12 | (0.05-0.19) | 0.1 | (0.07-0.13) | 0.07 | (0.06-0.08) | 0.41 | (0.27-0.62) |
| Distal Colon | 0.41 (0.19-0.76) | 19.7 | (11.1-40.7)^{a} | 0.55 | (0.50-0.78)^{b} | 0.43 | (0.38-0.70)^{b} | 0.53 | (0.21-1.34)^{b} |
| U. tract | 0.18 (0.16-0.46) | 1.5 | (0.51-1.54)^{a} | 0.19 | (0.17-0.39)^{b} | 0.25 | (0.21-0.28)^{b} | 0.13 | (0.06-0.19)^{b} |
| Kidney | 9.3 (6.4-11.5) | 52.8 | (28.52-61.6)^{a} | 17.4 | (11.3-30.4) | 2.7 | (2.04-16.1)^{b} | 3.9 | (2.85-8.09)^{b} |
| Lungs | 0.65 (0.52-0.73) | 537.7 | (402.5-735.5)^{a} | 12.6 | (10.2-15.5)^{a+b} | 6.4 | (3.9-19.3)^{b} | 5.3 | (2.4-8.4)^{a+b} |
| Trachea | 1.14 (0.61-1.67) | 28.2 | (14.3-50.2)^{a} | 0.71 | (0.34-1.59)^{b} | 1.11 | (0.25-3.96)^{b} | 1.1 | (0.41-1.44)^{b} |

Note.
SE—Surface epithelium,
LP—lamina propria,
NaB—sodium butyrate;
TBG—tri-butyrate glycerol;
PB—4-phenylbutyrate.
Data expressed as median with 25 and 75 percentiles in parentheses.
Healthy control rabbits are without any treatment.
One-way ANOVA was applied to compare between the different groups and when significant, post-hoc Holm-Sidak or Dunn's test was performed. The differences were significant when P < 0.05;
^{a}significant when comparing with healthy,
^{b}significant when comparing with infected.

Pharmacokinetics

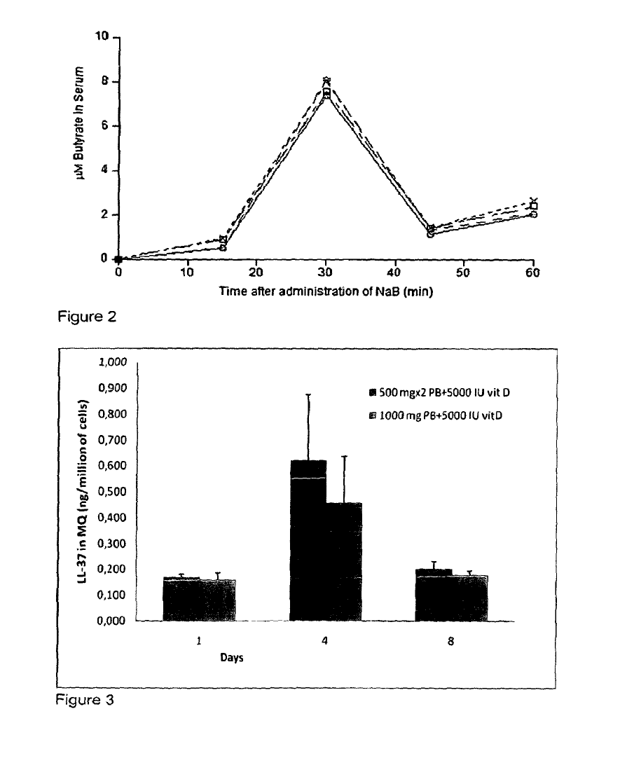

FIG. 2 shows the absorption curve of sodium butyrate in the rabbit after oral administration. The maximum concentration of butyrate in the blood is about 8 μM (Cₘₐₓ) after oral dosing of 15.6 mg of butyrate to each rabbit.

This is 50,000-fold lower than needed for maximum killing of both Shigella dysenteriae type 1 strain and Shigella flexneri strain which is achieved at 400 mM (44 mg/ml) (Raqib R et al (2006) PNAS, (103) 9178)

After oral treatment with NaB it was detected as butyrate in serum.

The functional importance of this result demonstrating a systemic effect was further substantiated by the findings that intravenous injection of NaB into infected rabbits also induced upregulation of CAP-18 in the rectal (6.5±1.8) and colonic SE (5.6±1.8). In a shigellosis model a 0.14 mmol/dose lead to 2 out of 3 rabbits recovering from shigellosis. Assuming a blood volume of 121 ml in the 2 kg rabbit the maximum concentration of NaB in the blood after IV administration would have been 1.2 mM.

The levels of CAP-18 were comparable to those noted in orally NaB treated rabbits (8.13±1 and 7.4±0.5 respectively) and were higher than in infected rabbits (4.9±1.1 and 3.8±1.8 respectively) as measured by ACIA scores.

The 0.14 mmol dose for the effective intravenous administration in rabbits (15.6 mg Na-butyrate or 26 mg Na-phenylbutyrate) translates to 2 mmol dose in the human. This would be equivalent to 222 mg of sodium butyrate or 372 mg of sodium phenylbutyrate intravenous dose in the human, in each case administered twice daily.

### Bactericidal Activity of Synthetic CAP-18 and PBA

To assess bactericidal activity of synthetic CAP-18 and PBA, an in vitro killing experiment was carried out with E. coli strain E2348/69 (EPEC). After an overnight incubation at 37° C., 97-99% killing of EPEC was observed at a concentration of 0.90 μM of CAP-18 (4 μg/mL) and 120 mM of PBA individually; a combination of 0.45 μM of CAP-18 (2 μg/mL) and 60 mM of PBA also showed maximal killing activity, which represented clear synergistic activity. The minimal inhibitory concentration of PBA for EPEC can be compared with the Cₘₐₓ of 1.2 mM after the administration of a 5 g dose. Note that dosing in Phase II TB study is 1 g daily with 5000 IU vitamin D.

Similar synergy (between the compounds of the invention, and the peptides which they induce) was also demonstrated using Shigella and PBA

As those skilled in the art are aware, E. coli (e.g. EPEC) infects the upper part of the gastro intestinal tract, the jejunum and the ileum, but not the colon or rectum. Thus the demonstration of induction of CAP-18 in the upper part of the gastrointestinal tract and the concomitant recovery of EPEC infected rabbits demonstrate a new utility for the compounds described herein in the treatment of upper GI tract infection.

### Example 2

### Phase I study: Expression of LL-37 in Macrophages from the Blood of Healthy Volunteers

Unless stated otherwise clinical materials were obtained from Fyrklövern, Scandinavia.

The present example demonstrates that treatment with PBA and vitamin D leads to expression of LL-37 in blood macrophages in humans. Furthermore, the same macrophages demonstrate improved efficacy in killing of TB bacteria in vitro.

A recent publication by Martineau et al (Lancet 2011; 377: 242-50) describes a Phase II study of TB patients treated with high dose vitamin D. The data described herein suggest that the combination of PBA and vitamin D would be more powerful in inducing antimicrobial peptides than either component alone.

A Phase II efficacy study of PBA and vitamin D as adjunct therapy in the treatment of TB is ongoing in Bangladesh.

The Phase I study of PBA and vitamin D was performed as follows:

Six healthy individuals were randomly assigned into two groups A and B, three in each group.
A. Received 500 mg PBA (twice daily) and 5000 IU vitamin D (once daily) for four days (d1-d4)
B. Received 1000 mg PBA (once daily and 5000 IU vitamin D (once daily) for four days (d1-d4)

PBA under the brand name Tributyrate was obtained from Fyrklövern Scandinavia AB, Sweden as 1 g enteric coated tablets. They were split into two parts as appropriate. Blood was sampled before first drug administration on day 1, after last drug administration on day 4 and then on day 8 after four drug-free days.

Expression of LL-37 and mRNA in Macrophages

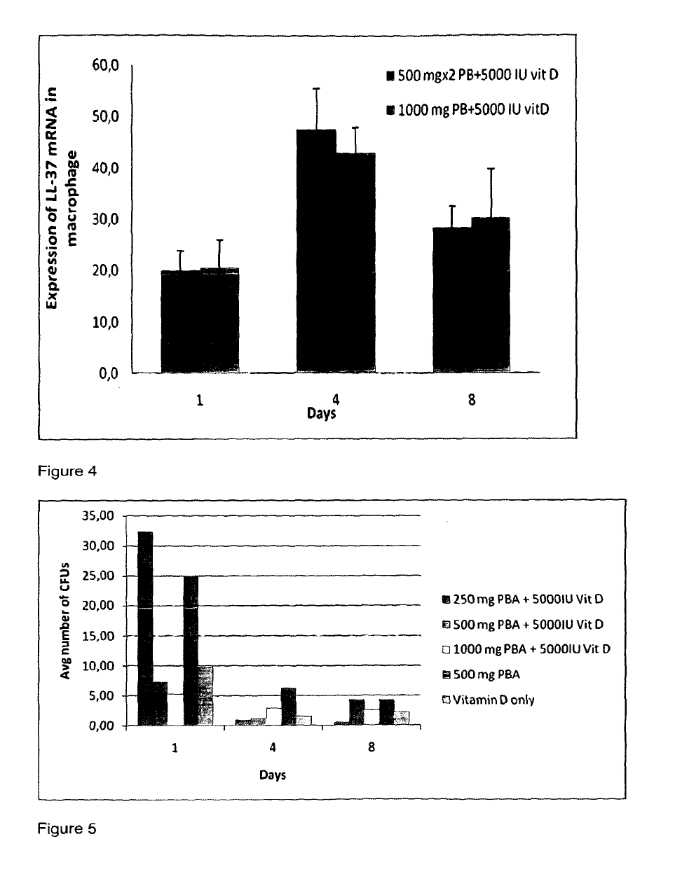

Macrophages were isolated and the expression of LL-37 determined by ELISA and rtPCR. The results are shown in FIGS. 3 and 4.

Killing of M. tuberculosis Bacteria in Macrophage Co-Culture

The killing of Mycobacterium tuberculosis was determined in a co-culture of macrophages from 15 healthy volunteers. The volunteers were randomly assigned to five groups that received the following treatment for four days with a subsequent 4 day treatment free period:
1. 250 mg PBA+5000 IU vitamin D
2. 500 mg PBA+5000 IU vitamin D
3. 1000 mg PBA+5000 IU vitamin D
4. 500 mg PBA
5. 5000 IU vitamin D

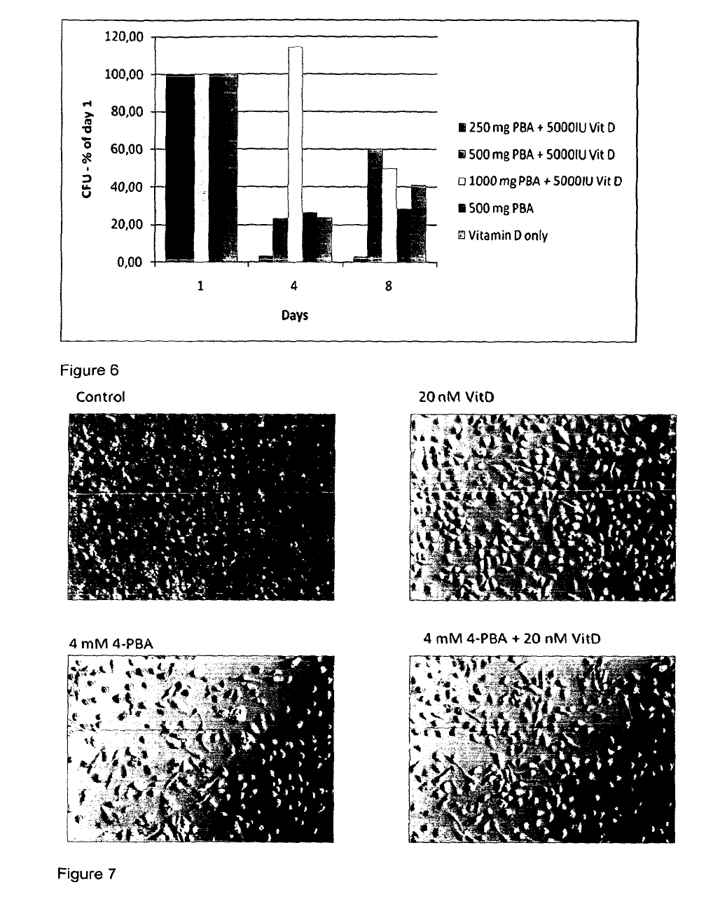

Blood was sampled before the first drug administration, after 4 days of treatment and at day 8, after a 4 day treatment free period. Macrophages were isolated from the blood samples. After cultivating the macrophages for 3 days the culture was infected with 25-50 CFU of M. tuberculosis/macrophage and grown further for 3 days xx days. Subsequently the culture was sampled and plated on 7H11 Middlebrook's medium. Plates were incubated at 35° C. for 25-28 days. Colonies were counted over transmitted light. Results are shown in FIGS. 5 and 6.

FIG. 5 shows that the average killing capacity of macrophages from samples of volunteer blood is highly variable before treatment starts. Thus the averages (n=3) in the five groups range from 5 to 32 CFUs. After four days of treatment the survival of bacteria is reduced in all groups which could be explained by the concomitant increase in production of LL-37 peptide and mRNA (see FIGS. 3 and 4). After four treatment-free days the survival of the bacteria does not return to the initial levels.

Survival of bacteria was reduced in all 15 individuals at day 4 relative to day 1 except one which explains the apparent increase in the group that received 1000 mg PBA+5000 IU VitD. Same level of killing of bacteria was observed in 9 individuals at day 8 relative to day 4 while an increase was observed in 6 individuals. All individuals demonstrated higher killing capacity at day 8 than before start of treatment, except one where there was no change.

Statistical comparisons cannot be made due to the small group size.

### Conclusion.

Samples taken from healthy individuals before start of treatment, after 4 days of treatment and after additional four days without treatment show that 4-day treatment with the combination of PBA and vitamin D increase the expression of both LL-37 mRNA and protein. Furthermore, after a 4-day treatment-free period the mRNA levels are still elevated and the bacterial killing capacity has not returned to pre-treatment levels. The data suggests that oral administration of PBA and/or Vitamin D act systemically to increase the levels of antimicrobial peptides in cells including blood cells. This suggests that PBA treatment with or without vitamin D can be used to treat systemic infections including TB or Pseudomonas.

### Example 3

### Demonstration of Killing of Pseudomonas Bacteria Grown on Human Bronchial Epithelial Cell Line, VA10, in the Presence of Dead Bacteria, PBA and Vitamin D

A series of studies were performed to assess the effects of inducers of antimicrobial peptides (LL-37 and β-defensin 1) on VA10 epithelial cell line. In addition, the effects of inactivated Pseudomonas bacteria on secretion of antimicrobial peptides were assessed.

VA10 cells were grown in BEGM medium (Bronchial Epithelial Growth Medium). VA10 cells were induced by adding PBA and/or vitamin D to the medium. Untreated control was included. After 48 h induction the culture was infected with low dose of Pseudomonas aeruginosa (strain PA01) along with freeze-thaw-treated bacteria. After 24 hrs of cultivation the culture wells were visually inspected. Dense culture of bacteria was found in control wells, but the wells of cells that had been induced with 20 nM vitamin D, 4 mM PBA or combination of 20 nM vitamin D and 4 mM PBA contained clear medium (FIG. 7).

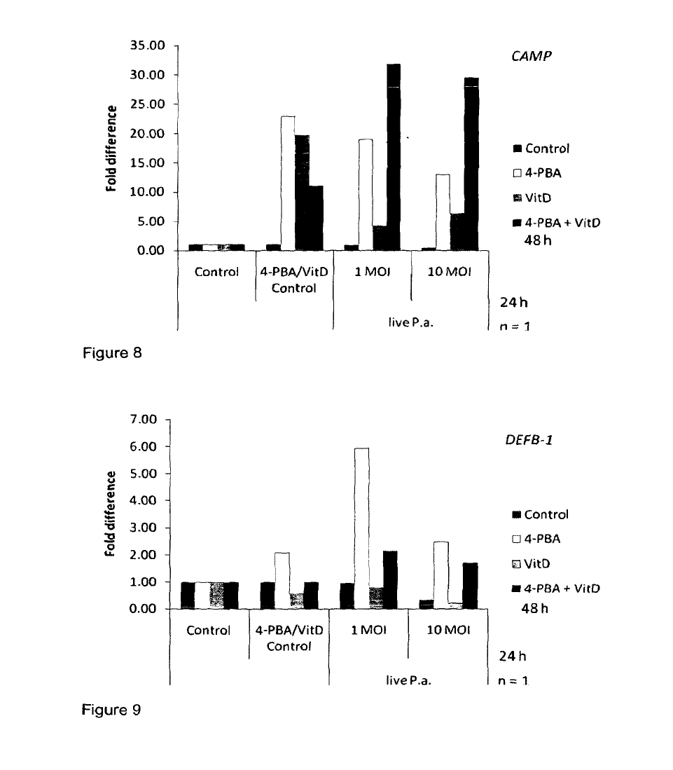

In addition to the visual inspection the degree of induction of CAMP mRNA, that codes for LL-37, and DEFB-1, that codes for δ-defensin 1, was determined by rtPCR. The results are shown in FIGS. 8 and 9.

The results shown in FIGS. 8 and 9 indicate a powerful stimulation of CAMP expression as a result of incubation with known stimulants. Additional stimulation results from incubation with inactivated Pseudomonas bacteria. Maximal stimulation was obtained with 48 hr incubation with the combination of 4 mM 4-PBA and 20 nM VitD with subsequent incubation with inactivated bacteria.

Similarly, the DEFB-1 expression can be stimulated up to six-fold by incubating first with 4-PBA and subsequently with low-dose inactivated Pseudomonas bacteria.

The stimulated expression of antimicrobial peptide mRNA, CAMP and DEFB-1, may explain the results observed in the cell cultures where survival of the VA10 cells coincides with high expression of CAMP and DEFB-1. Cells that were not stimulated prior to being infected demonstrated cloudiness in the well (FIG. 8, ‘control’). The cloudiness suggests uninhibited growth of the bacteria, while the stimulated cultures had clear medium and healthy VA10 cells suggesting killing of bacteria by secreted antimicrobial peptides.

In a further experiment, VA10 respiratory epithelial cells were cultivated for 6 days before being stimulated for 48 h with 4-PBA+VitD with or without dead PAO1 Pseudomonas aeruginosa 0-1 bacteria (PAO1). Subsequently the cells were challenged for 24 h with 10 MOI (multiplicity of infection) Pseudomonas aeruginosa 0-1 bacteria before harvesting the bacteria, plating them on medium and counting the colony forming units. Number of CFUs is expressed as % of control that was not stimulated with PBA and Vitamin D or dead bacteria before being challenged. The Table below shows data from two independent experiments:

% (relative number of CFUs)

| Stimulation | Experiment 1 | Experiment 2 |
|---|---|---|
| Control | 100 | 100 |
| Vit D and PBA | 43 | 72 |
| Vit D and PBA and dead P.a. | 39 | 56 |

### Example 4

### Demonstration of killing of Haemophilus influenzae and Moraxella catarrhalis Bacteria

### Inhibition Zone Assay

Against Haemophilus influenzae

| CAP-18 | Zone diameter | LL-37 | Zone diameter |
|---|---|---|---|
| Con | (mm) | Conc | (mm) |
| 5 uM | 0 | | |
| 10 uM | 5.4 | | |
| 25 uM | 7.65/8.7 | | |
| 50 uM | 9.55/10.5 | 50 uM | 0 |
| 100 uM | 10.85 | 100 uM | 7.75 |
| 200 uM | 12.25 | 300 uM | 13.3 |

Against Moraxella catarrhalis

| CAP-18 | Zone diameter | LL-37 | Zone diameter |
|---|---|---|---|
| Con | (mm) | Conc | (mm) |
| 5 uM | 5 | | |
| 10 uM | 6/7.2 | 10 uM | 5.4 |
| 25 uM | 8.2/9.1 | 25 uM | 6.3 |
| 50 uM | 9.5 | 50 uM | 7.45 |
| 100 uM | 11.35 | 100 uM | 8.9 |
| 200 uM | 13.2 | | |

This in vitro killing of respiratory pathogens, Haemophilus influenzae and Moraxella catarrhalis by CAP-18 further supports the utility of the compounds of the present invention as methods of inducing antimicrobial peptides and enhancing the innate epithelial barrier of the respiratory system, which may be impaired by these respiratory pathogens.

1. A method of treatment or prophylaxis of an infection caused by a pathogen in a patient in need of the same, which method comprises oral or intravenous administration to the patient an effective amount of a compound of formula (I) 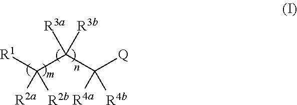
wherein
Q represents —COOH, —COOR⁵, or a pharmaceutically acceptable salt of —COOH;
R¹ represents an unsubstituted aryl group, hydrogen, a linear or branched unsubstituted or substituted saturated or unsaturated alkyl group with 1 to 10 carbon atoms, halide, amino, hydroxyl, carbonyl, or a substituted aryl group;
R^{2a}, R^{2b}, R^{3a}, R^{3b}, R^{4a} and R^{4b}, if present, each independently represent hydrogen, halide, amino, hydroxyl, carbonyl, a linear or branched substituted or unsubstituted saturated or unsaturated alkyl group with 1 to 10 carbon atoms, or a substituted or unsubstituted aryl group; and/or
R^{3a}, together with an adjacent R^{4a} or R^{2a}, may represent a carbon-carbon π bond; and/or
R^{3b}, together with an adjacent R^{4b} or R^{2b}, may represent a carbon-carbon π bond;
m and n are each independently 0 or 1;
R⁵, if present, represents a linear or branched substituted or unsubstituted saturated or unsaturated alkyl group with 1 to 10 carbon atoms, a substituted or unsubstituted aryl group, a triglyceride moiety —CH₂CH(OC(═O)R⁶)CH₂(OC(═O)R⁷), or a diglyceride moeity —C(═O)OCH₂CH(OC(═O)R⁶)CH₂OH or a salt thereof; and
R⁶ and R⁷, if present, independently represent hydrogen, a linear or branched substituted or unsubstituted saturated or unsaturated alkyl group with 1 to 10 carbon atoms or a substituted or unsubstituted aryl group.
wherein:
(i) said infection is selected from infection of the lung, trachea, urinary tract or kidney, upper GI tract and\or blood
and\or
(ii) said pathogen is selected from: Mycobacterium tuberculosis; Pseudomonas bacteria; Haemophilus influenzae; Moraxella catarrhalis;
and in each case wherein the effective amount is an oral dose of between 500 and 4000 mg/day, or an intravenous dose of between 200 and 1000 mg/day such as to boost innate antimicrobial activity in the patient

2. A method as claimed in claim 1 wherein the compound is selected from:
(i) 4-phenylbutyric acid or a salt of 4-phenylbutyrate,
(ii) butyric acid or a salt of butyrate, such as sodium butyrate (compound IIb)
(iii) glyceryl tributyrate (TBG)
(iv) 2-methyl-3-phenylpropionic acid or a salt of 2-methyl-3-phenylpropionate.

3. A method as claimed in claim 2 wherein the compound is sodium 4-phenylbutyrate.

4. A method as claimed in claim 2 wherein the compound is administered in combination with Vitamin D.

5. A method as claimed in claim 2 wherein the compound is administered such as to boost the innate antimicrobial activity in the lung, trachea, urinary tract or kidney, jejunum, ileum.

6. A method as claimed in claim 2 wherein said infection is tuberculosis and\or the pathogen is Mycobacterium tuberculosis.

7. A method as claimed in claim 6 wherein said patient is immunocompromised, and is optionally HIV positive.

8. A method as claimed in claim 6 wherein the compound is a salt of 4-phenylbutyrate, and the compound is administered in combination with Vitamin D.

9. A method as claimed in claim 2 wherein the said infection is an infection of the blood.

10. A method as claimed in claim 9 wherein the compound is administered such as to boost the innate antimicrobial activity by inducing anti-microbial peptides in white blood cells.

11. A method as claimed in claim 2 wherein the said infection is a respiratory infection of the respiratory airways or lungs.

12. A method as claimed in claim 11 wherein said infection is secondary infection which is associated with dysenteric or cholera-like diarrhoea, optionally arising from shigella infection.

13. A method as claimed in claim 12 wherein said secondary infection is viral or bacterial.

14. A method as claimed in claim 13 wherein said secondary infection is pneumonia or meningitis.

15. A method as claimed in claim 11 wherein the said infection is a lung infection and said pathogen is Pseudomonas bacteria.

16. A method as claimed in claim 15 wherein said pathogen is Pseudomonas aeruginosa and said compound is 4-phenylbutyric acid or a salt of 4-phenylbutyrate which is administered in combination with Vitamin D.

17. A method as claimed in claim 11 wherein said pathogen is Haemophilus influenzae and Moraxella catarrhalis.

18. A method as claimed in claim 2 wherein said infection is a kidney or urinary tract infection and said compound is TBG.

19. A method as claimed in claim 2 wherein the treatment or prophylaxis of the infection comprises:
(1) administration to the patient of an antibiotic for 1 or 2 days with or without a compound of formula (I); followed by
(2) administration to the patient of an effective amount of a compound of formula (I) for a further 2, 3, 4, 5 or more days.

20. A method as claimed in claim 2 wherein the effective amount is between 500 and 2000 mg/day 4-phenylbutyric acid or a salt of 4-phenylbutyrate given orally.

21. A method as claimed in claim 2 wherein the effective amount is between 200 and 700 mg of sodium butyrate or 600 and 1000 mg of sodium phenylbutyrate given intravenously.

22. A method as claimed in claim 2 wherein the effective amount is between 3000 and 4000 mg/day TBG given orally.

23. A method as claimed in claim 2 wherein the daily dosage of the compound of formula (I) is split into doses given 2 or 3 times daily.

24-27. (canceled)

28. A method as claimed in claim 1 wherein said infection is a lung infection and said pathogen is Pseudomonas bacteria.
